(19) United States
(12) Patent Application Publication
(10) Pub. No.: US 2020/0226689 A1
(43) Pub. Date: Jul. 16, 2020

(54) HEALTHCARE PRACTICE MANAGEMENT SYSTEM AND METHOD THEREOF

(71) Applicant: Vericle Corporation, Clearwater, FL (US)

(72) Inventors: Yuval LIROV, Clearwater Beach, FL (US); Erez LIROV, Morganville, NJ (US)

(21) Appl. No.: 16/248,725

(22) Filed: Jan. 15, 2019

(51) Int. Cl. G06Q 40/08; G16H 10/60; G06F 16/2457

(52) U.S. Cl. CPC G06Q 40/08; G06F 16/24578; G16H 10/60

(57) ABSTRACT

A healthcare practice management system and method that minimizes float healthcare insurance and related type claims. The healthcare practice management system and method reduces overall float by creating a task list so that problematic claims are addressed, so that overall float in the system is minimized and managed.

### FIELD OF THE INVENTION

The present invention relates to a practice management framework for healthcare practice management. The present invention relates to systems, methods and non-transitory subject matter for reducing payor float in a computer-implemented health insurance billing platform and software.

### BACKGROUND OF THE INVENTION

Healthcare institutions struggle to receive payments for their services from insurance companies and payors of medical services. There is a constant battle between healthcare institutions (“payees”) and insurance companies, such as Blue Cross Blue Shield and governmental organizations, such as Medicare and Medicaid, (“payors”) to process claims and transmit payment for services provided.

Payees work diligently process claims so to timely collect payment from payors. However, delays often are present in transmitting and collecting insurance premiums from payors that transmit payment. Payors, such as insurance companies, do not pay out all the money they collect right away. Rather, an insurance company will collect money in the form of premiums from its clients, invest that money, and then pay out claims as needed at some future date. The difference between premiums collected and claims paid out is insurance float or float.

In practice, healthcare institutions have large numbers of clients and submit thousands of claims to various payors. Tracking the claims and payments and having payments processed is extraordinarily complex, as insurance companies have a built-in incentives not to pay and to maximize float, as it gives maximum amounts of time to invest money from clients. Furthermore, insurance companies intentionally fail to make payments or make underpayments in order to increase float and have additional time in which to invest float funds.

For healthcare institutions, there is an incentive to minimize insurance float. However, tackling this task is difficult as it is often difficult to determine which insurance claims to target and to follow up on to have paid. The total number of decision possibilities in a typical healthcare institution practice is comparable to the number of possibilities in a chess game. A typical healthcare practice owner and/or practice management staff do not have the mental wherewithal to manage such complexities daily, and it is often impossible to choose the proper claim to work on during the day in order to have it paid so to reduce overall payor float owed to the healthcare institution.

For example, a healthcare institution might have a patient roster of 500 patients. Each patient has their own payor which has its own set of rules and complicated processes. In this example, complexity exists as processing the order of claims is often extremely laborious and time consuming.

For a larger practice, say 10 offices each containing 500 patients; complexity reaches thousands of decision points and it is often impossible to target the claims to focus on to reduce payor float; as workflow reaches astronomical hyper-complexity.

Ideally, staff in healthcare institutions need to choose their best actions and perform each quickly, accurately, timely, and pressing the envelope of their own limitations and to target the claims to reduce payor float. Given the astronomical number of potential complex choices, it is mentally impossible to ensure an optimal decision for each one.

This growing complexity negatively affects a practice in multiple ways. It exacerbates practice management challenges by inviting more errors which domino into errors causing more delays which adds to overall complexity and thus creating a vicious downward productivity cycle.

As staff make more mistakes in processing claims, often payor float increases as payor float delay payment in order to maximize profits.

Current methods and systems are not designed with the goal to minimize payor float and with the aspects of complexity and payor adversity in mind. As a result, practice owners and managers must analyze reports, consider action alternatives, and make the best management decisions based on their personal experience and mental ability to compare the alternatives in mind, rather than taking a systematic and approach to reducing insurance float.

The only alternative is memory-management which is humanly impossible; hence very few practice owners thrive in healthcare and healthcare insurance business.

Accordingly, it is an object of the present invention to provide systems and methods to solve the problems set forth above.

### SUMMARY OF THE INVENTION

It is an object of the instant invention to provide systems and methods to reduce insurance float.

It is an object of the invention to provide systems, methods and non-transitory computer readable medium that execute instructions to reduce insurance float. In certain embodiments, the instructions are stored on a memory and are executed by a computer processor.

It is an object of the invention to provide systems, methods and non-transitory computer readable medium that execute instructions to provide workflow management and a task list so that staff are able to effectively allocate their resources to process claims and reduce insurance float.

Objects of the invention are achieved by providing a computer-implemented health insurance billing system, comprising: a memory comprising computer executable instructions; and a processor coupled to the memory and configured by the computer executable instructions, the processor configured to:
(1) access at least one database having patient claim data;
(2) rank the patient claim data to create a ranked list of patient claim data; and
(3) generate a list of action items based upon the ranked list of patient claim data, wherein the list of action items are sorted according to payor float of each of the patient claim data items,
wherein steps (2)-(3) are completed on the processor.

In certain embodiments, the computer-implemented health insurance billing system minimizes overall payor float.

In certain embodiments, the patient claim data is sorted by payor float of each of the patient claim data items.

In certain embodiments, the payor float is normalized by a float factor.

In certain embodiments, the float factor includes payor information, the payor information sorted by payor difficulty in making payments and payment delay.

In certain embodiments, the float factor is at least partially sorted by an amount of each of the unpaid patient claim data items.

In certain embodiments, the float factor includes a weighting system, such that the weighting system is adjusted based upon payor data.

In certain embodiments, the system includes heuristic learning, wherein the system is configured to adjust the weighting system based upon heuristic learning.

In certain embodiments, the float factor is adjusted based upon batch processing such that patient claim data items are sorted into groups and combinations of groups contribute to payor float.

In certain embodiments, the float factor is configured based upon specific rules, the rules are followed to create the ranked list of patient claim data.

In certain embodiments, the float factor is adjusted based upon CPT codes and in-network reimbursement fees.

In certain embodiments, the computer-implemented health insurance billing system provides a workflow of action items for each day.

In certain embodiments, the workflow of action items for each day minimizes overall payor float.

In certain embodiments, the system automatically provides a list of top 10 action items to work on for each day.

In certain embodiments, the system automatically provides a list of action items to work on for each day and delegates them to individual staff at a healthcare institution

In certain embodiments, the system automatically generates a task list on a work bench for multiple users.

In certain embodiments, the at least one database is selected from a group consisting of patients, providers, practice management staff, claims, claim validation rules, tasks, process description, or a combination thereof.

In certain embodiments, the system is in communication with the payor in order to send claims, appeals, notifications and receive notifications and payments.

Other objects of the invention are achieved by providing a computer-implemented method for minimizes overall payor float in a health insurance billing system, the method comprising:
(1) accessing at least one database having patient claim data;
(2) ranking the patient claim data to create a ranked list of patient claim data; and
(3) generating a list of action items based upon the ranked list of patient claim data, wherein the list of action items are sorted according to payor float of each of the patient claim data items,
wherein steps (2) and (3) are completed on a processor.

In certain embodiments, step (2) includes a custom software/algorithm based on game theory.

Other objects of the invention are achieved by providing a non-transitory computer readable storage medium storing a computer program product for minimizing payor float in a healthcare insurance billing system, the non-transitory computer readable storage medium comprising: computer executable instructions and data, the computer executable instructions able to execute a computer program able to:
(1) access at least one database having patient claim data;
(2) rank the patient claim data to create a ranked list of patient claim data; and

(3) generate a list of action items based upon the ranked list of patient claim data, wherein the list of action items are sorted according to payor float of each of the patient claim data items,
wherein steps (2)-(3) are performed on a processor.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a flow chart depicting a computer-implemented method and system of the present disclosure.

FIG. 2 is a second flow chart providing action items for the computer system and method.

FIG. 3 is a flowcharts providing various options through which various tasks are performed.

FIG. 4 is a flowchart providing tasks in a work bench.

FIG. 5 is a flowchart/tree of various possible actions.

FIG. 6 is a flowchart of a method for a Computer-Assisted Health Insurance Billing To Minimize Payer's Float.

FIG. 7 is a flowchart of another method for a Computer-Assisted Health Insurance Billing To Minimize Payer's Float.

FIG. 8 is a flowchart of another method for a Computer-Assisted Health Insurance Billing To Minimize Payer's Float.

FIG. 9 is a flowchart showing underpayment of an insurance provider such that float is maximized.

### DETAILED DESCRIPTION OF THE INVENTION

In the following description, numerous details are set forth for the purpose of explanation. However, one of ordinary skill in the art will realize that the invention may be practiced without the use of these specific details.

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary medical devices for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The embodiments are described more fully hereinafter with reference to the accompanying drawings, in which embodiments of the inventive concept are shown. In the drawings, the size and relative sizes of layers and regions can be exaggerated for clarity. Like numbers refer to like elements throughout. The embodiments can, however, be embodied in many different forms and should not be construed as limited to the embodiments set forth herein. Rather, these embodiments are provided so that this disclosure will be thorough and complete, and will fully convey the scope of the inventive concept to those skilled in the art. The scope of the embodiments is therefore defined by the appended claims.

Reference throughout the specification to “one embodiment” or “an embodiment” means that a particular feature, structure, or characteristic described in connection with an embodiment is included in at least one embodiment of the embodiments. Thus, the appearance of the phrases “in one embodiment” or “in an embodiment” in various places throughout the specification is not necessarily referring to the same embodiment. Further, the particular feature, structures, or characteristics can be combined in any suitable manner in one or more embodiments.

This application incorporates by reference U.S. application Ser. No. 14/665,502 filed on Mar. 23, 2015. The contents of this application are incorporated by referenced herein in their entirety.

### Float

Insurance companies and payors earn profits in multiple ways. A well-known insurance company business model practice relies on collecting more in premiums than is paid out in claims. A lesser known practice relies on investing what is referred to as “float”. Float is the available reserve of funds on hand at any given time; meaning, premiums collected but not yet paid out in claims. Insurance companies and other payors capitalize on the fact that they have access to a reserve of funds available between the time they receive payment and when they are to pay out potential claims. The amount of time that the insurance company can hold onto said funds is proportional to the return; and therefore, the value of such an asset. The longer the duration, the more return that can be generated using this float.

Not surprisingly, this results in payors having a disincentive toward paying out claims in a timely manner. Insurance companies enact strict rules and requirements that must be met before any claim funds are paid out. Every delay furthers the payor's hold onto the float. The slower the pay-out process goes the more float the payor accumulates over time. Additional unnecessary delays are caused by provider errors and neglect. Providers risk making errors at every stage of their workflow extending the time until a payee receives payment.

### System Objectives

It is contemplated that the instant inventive system and method reduces practice management complexity by analyzing patient claims and creating a task list to minimize payor float.

It is contemplated that the instant inventive system and method identifies and selects staff members to perform tasks in order to reduce payor float.

It is contemplated that the instant inventive system and method identifies and selects, that is “delegates” tasks to staff members, providing them with instructions as to the best next workflow move and necessary information for accomplishing the task more efficiently, and for the best next allocation of tasks resulting in highest presenting the staff member(s) with the next best workflow move for that staff member, and providing all the relevant information (and ONLY the relevant information) to make that workflow move a success.

Complexity of healthcare practice management extends beyond human ability to manage it on paper and it grows quickly in step with the addition of new patients. Payors complicate the practice management challenge even more as they introduce an “adversity element” to the overall system because they keep the profit derived from investing the float (the difference between premiums collected and payments made) in financial markets.

The adversity element is, for example, an underpayment or complexity created by a payor in order to maximize float.

### Complexity Created by Payors

Payors like healthcare institutions to make mistakes in processing claims in order to increase payor float. Payors often do not pay out claims that have spelling mistakes or are not in compliance with payor requirements.

Typical types of complexity created by payors involves underpaying claims, providing in-network and out of network rates, providing customized group rates and other such payments in order to increase complexity of payments, so as to increase payor float.

Payors like systems and staff making wrong choices to reduce float, and often increase their own complexity of systems in order to increase float. By increasing float, payors receive an interest free loan that they are able to invest and keep the profits that are made.

### Advantages of system

Embodiments of the invention involves systems and methods that evaluate float possibility & rank orders by highest float possible. Possibilities are provided in the work bench.

The system provides a ranked order of claims to work on and does so automatically, so that staff can target problematic claims and reduce the amount of float.

The system and method is able to handle thousands of claims and unpaid claims and sorts through various choices that leads to high float.

The system has the following advantages:
1) Understands float;
2) Translates float into a zero sum game;
3) Translates into prioritization of specific action items; and
4) Figures out workflow for a workday (best moves in float minimization game).

Embodiments of the system involve creating a tree of possible actions and several possibilities of each date. The system computes overall float for the next move (proxy is the float).

In certain embodiments, the system evaluates the unpaid amount of each claim, which is the claim value minus the amount paid and calculates the sum of unpaid amounts of each claim.

The system sums the options for each claim and provides a potential float reduction (PFR) for each claim. The items with the highest PFR values are worked on first, so that the overall float of the system is minimized.

### Weighting Factors

In certain embodiments, the system uses a weighing factor for each different payor in order to calculate a potential float reduction (PFR) for each item. For example, Medicare is an easy payor that often pays while insurance companies are difficult payors that often underpay. In this example, the delay of Medicare is 14 days to transmit payment while others have a 28-day delay.

In certain embodiments, the system applies a weighting factor to calculate float of each item and raises the priority of payors with longer delay. Thus, the value of each claim is normalized by a float factor in order to computer a normalized potential float reduction (PFR) for each item.

### Batch Processing

In certain embodiments, the system groups claims that are unpaid into batches. The system assigns more weight to correct a group of payments, such that if a group of payments are paid it reduces overall system float.

In an example, if there is a spelling mistake in a patient name, then the claim is not paid. Often spelling mistakes occur in a group of claims so the entire group of claims are unpaid. The system provides a weighting factor and assigns a higher weighting factor when correcting a groups of payments. The system clusters groups of possibilities in a batch process, so that the claims connected to the batch are assigned a higher weighting factor, and thus a higher PFR value.

In certain embodiments, combinations of lines of attach carry more weight. Heuristic rules to help estimate value of float with specific rules (such as add float together for groups of claims) which associate with cluster as single move.

In certain embodiments, batch processing of claims helps provide various claims with higher PFR value.

### Heuristic Learning

In certain embodiments, the system includes heuristic learning, wherein the system is configured to adjust the weighting system based upon heuristic learning. The system involves rules, such that the system is able to identify various spelling mistakes and provide a potential float reduction (PFR) adjustment based upon previously corrected items.

In other examples, the system has machine learning such that the system is able to learn from choices made by human operators, such that successful claims paid can adjust the potential float reduction (PFR) of each claim, as well as the float factor and weighting factor.

In certain embodiments, the system has various rules to expedite process claims to reduce float. In certain embodiments, there are 2 million rules and there are expedited rules that help process claims.

### Weighting Factor Calculation

In certain embodiments, the system includes a complex process in order to compile weighting factors. The system looks at underpayments, types of payors, In-network vs. out of network rates, and other considerations in order to calculate a weighting factors, which is then applied to each claim to obtain a PFT. Each item is then ranked by PFR in order rank the items, so that a ranked list of items can be compiled and worked on to minimize system float.

### Underpayment

In certain embodiments, the system accounts for underpayment of various claims. Underpayment is a delay tactic often employed by payors in order to partially pay claims, while then keeping the float funds for additional time in order to increase float.

In certain embodiments, the system is able to track and focus on underpayments made and increased the float factor based upon underpayments made. This then increases the priority of claims that have been underpaid, thus increasing their PFR and causing these payments to be worked on by staff to reduce overall system float.

In certain embodiments, the system uses machine learning and heuristic learning to identify patterns of underpayment. If a pattern of underpayment is recognized, then the normalized float factor is increased to give priority to types of claims that have consistent underpayment.

### Additional Complexity Factors

In certain embodiments, underpayment is a result of use of system codes where payors only pay up to a certain amount for codes for certain procedures.

Certain payors include CPT codes and corresponding tables whereby each code is linked to a maximum value paid out by the payor. The CPT codes must match the right diagnosis of the patient and the correct procedure and payors only make payments for items where all the information is correct.

Adding further complexity to this system is that there are different CPT codes for both in-network and out of network procedures, and payors often pay different rates for different procedures. Moreover, often payors strike deals with certain healthcare institutions based upon volume, which leads to additional complexity in codes and payments made.

Additional complexity results from codes having maximum payment amounts associated with them, such that if the healthcare institution charges more than what is allowed according to the code, then claims are either underpaid or not paid at all.

### Operation of System

In certain embodiments, the system includes a practice position that is defined as a sum of its payer positions. The total practice float is the sum of its floats for each payer. Every payer's response modifies practice position and its float. The new position defines a number of potential responses. Each potential response is evaluated using an evaluation function that estimates the resulting Float of the entire position across. The system:
1) prunes the list of all possible positions for evaluation (Float) to a much shorter list of most likely candidates to accelerate the position evaluation process, which would be otherwise prohibitively time consuming;
2) compares the potential position values on the pruned list and selects the next best position that reduces the float the most; and
3) places the move that defines that position on the workbench of the staff member that is responsible for that function.

### Figures

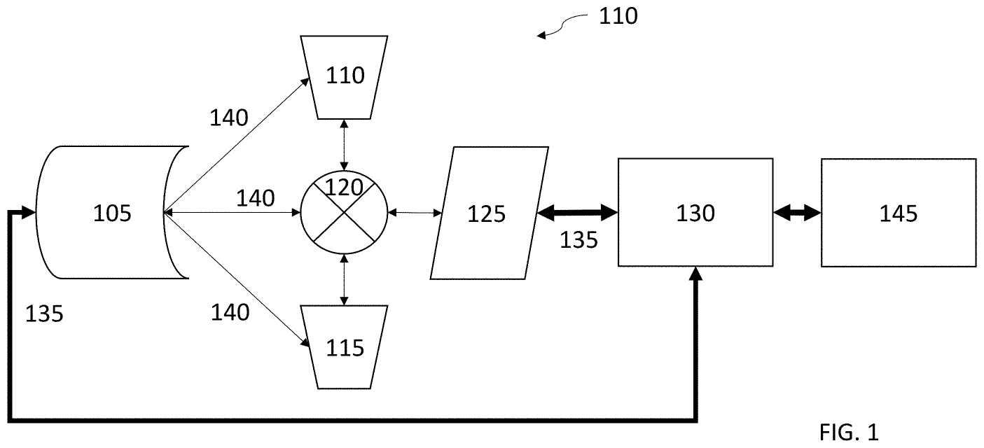

Referring to the figures, as depicted in FIG. 1, an embodiment of the instant invention (100) includes various databases: patients, providers, practice management staff, claims, claim validation rule database, tasks, and process descriptions (policies, flowcharts, and checklists) (105).

As shown, each staff member (110) and each provider (115) has an individual task and claim workbench (120), which must be periodically checked for presence of new items (125). A new item (125) may be bi-directional connectivity and communication with and to payors to send claims, appeals, receive notifications, authorize/send payments, and the like.

A selection or delegation algorithm (130) continuously iterates (135) through all the databases (105) and delegates (140) the best workflow action for each workbench (120).

A practice position is defined as a sum of its payor positions. The total practice float is the sum of the floats for each payor. Every payor's variable response modifies practice position and its float. The new position defines a number of potential responses. Each potential response is evaluated using an evaluation function (145) that estimates the resulting float of the entire position across each workbench (120).

The float calculation function (130) via the evaluation function (145) compares the potential position values on the pruned list of possible positions for evaluation; identifies and then selects the next best position that most reduces the float.

The float calculation function (130) prunes the list of all possible positions for evaluation (float) to a much shorter list of most likely staff member(s) (110) and/or provider(s) (115) to accelerate the position evaluation process (120), which would be otherwise prohibitively time consuming; and places the workflow move that defines that position in the workbench (120) of the staff member (110) responsible for that function.

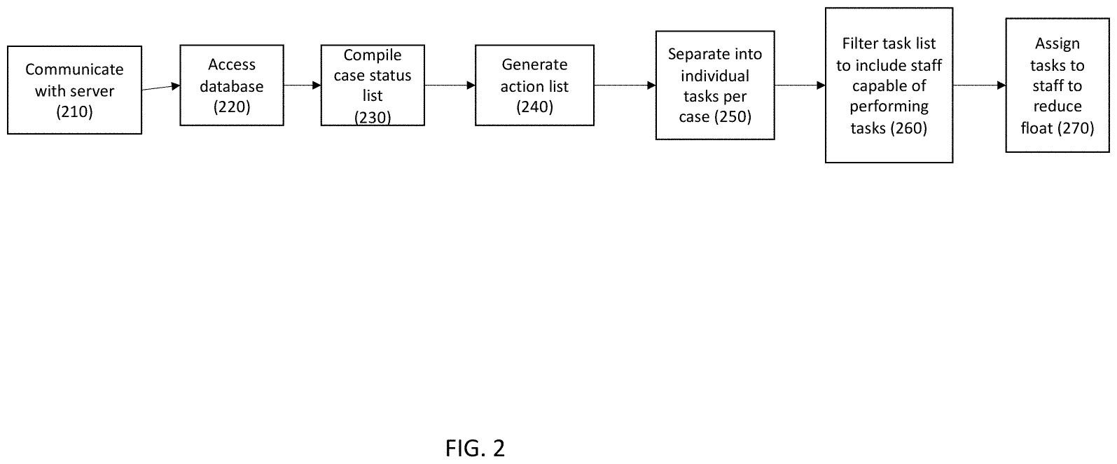

FIG. 2 shows a flowchart of an embodiment of the invention. In FIG. 2, the computer process communicates with a server (210) an accesses database (220). The processor compiles a case status list (230) and generates an action list (240). The processor executes instructions in memory in order generate a ranked list of items in order to reduce the overall system float of the system.

FIG. 2 shows additional stems of separating the tasks into individual cases (250), expanding the task list to include all staff members that can perform the task (260) and assigning tasks to staff members to reduce float (270).

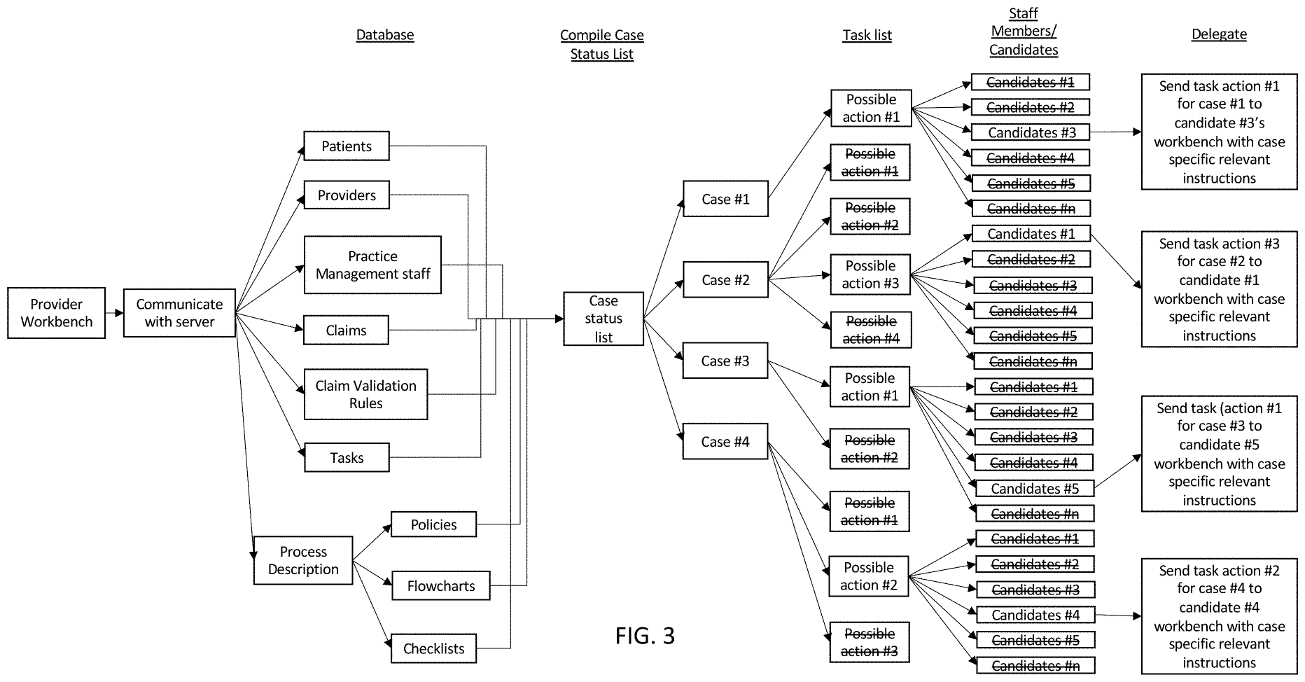

FIG. 3 shows a workbench and a task list showing various databases and action items and compiling a task list of cases and items across a workbench to various staff members/candidates to complete the tasks. The system complies the workflow and uses rules for claim validation and weighting factors in order to assign the tasks to staff/candidates for task completion in an organized and simplified manner.

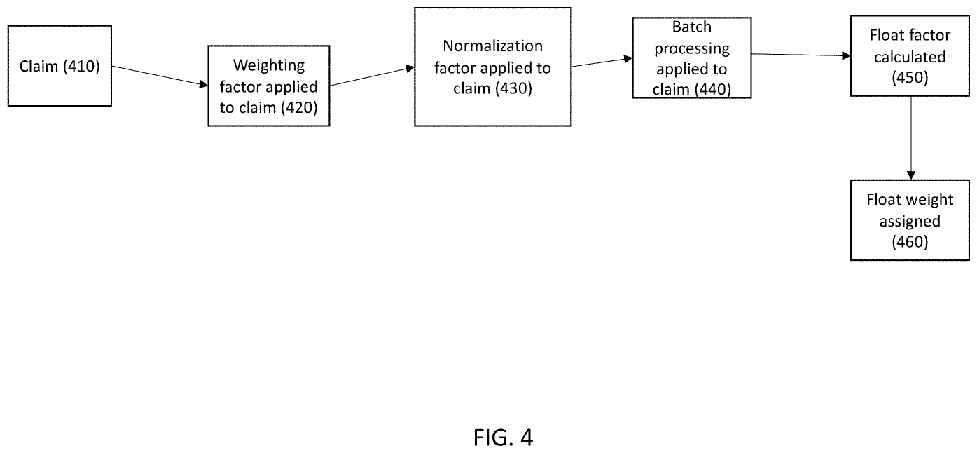

FIG. 4 shows a claim being given a float weight. The system and methods provide a weighting factor (420), normalization factor (430), batch processing factor (440) in order to calculate a float factor (450). The claim is then provided a float weight (460) and then the claim is placed in a ranked list according to float.

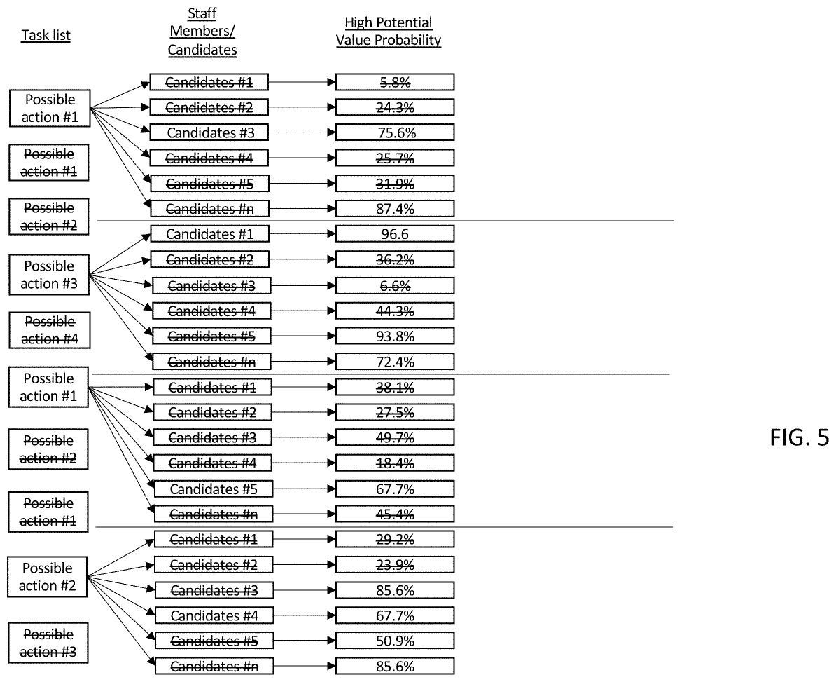

FIG. 5 shows a chart providing various tasks and possible actions and sorting the tasks based upon the potential value/probability to reduce float.

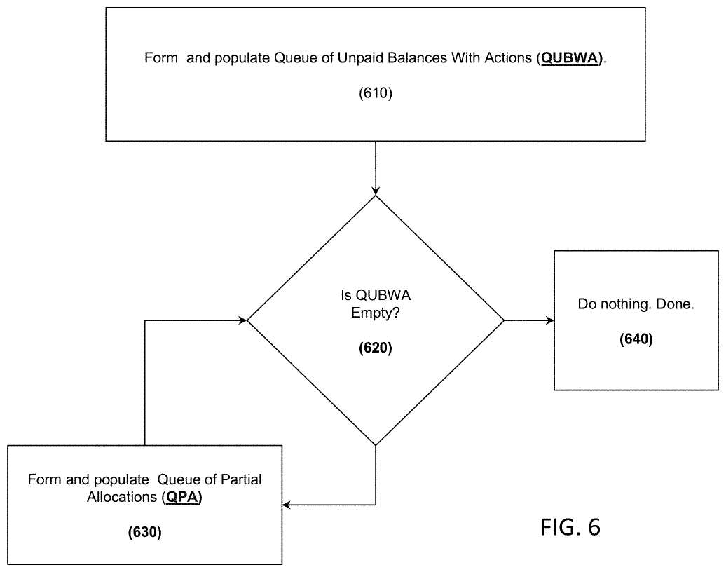

FIG. 6 illustrates a float chart of a method for a Computer-Assisted Health Insurance Billing To Minimize Payer's Float. In FIG. 6, the processor forms an populates a Queue of Unpaid Balances With Actions (QUBWA) (610). The processor checks to see if the QUBWA is empty. If not, then it forms and populates a que of partial allocations (QPA). This process is reiterated until a ranked task list is created.

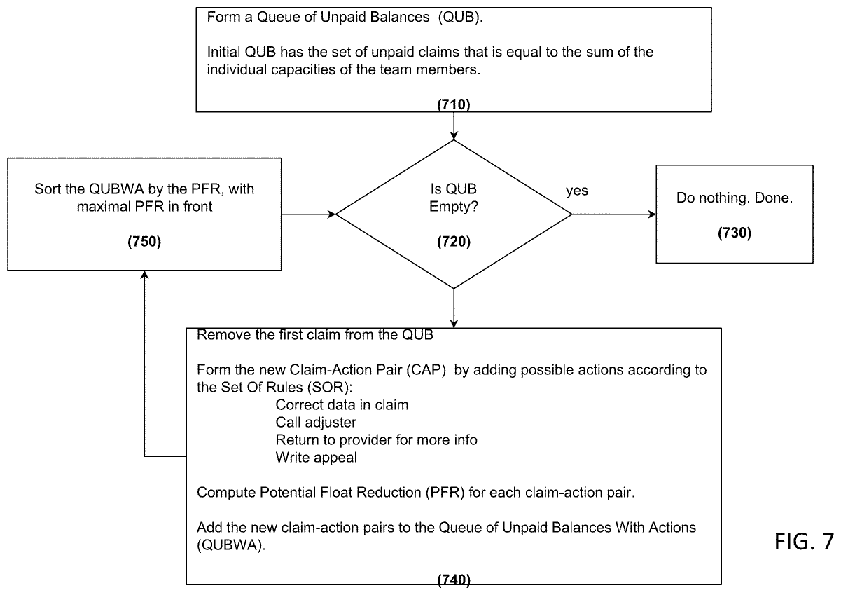

FIG. 7 shows a float chart of another method for a Computer-Assisted Health Insurance Billing To Minimize Payer's Float. In FIG. 7, the processor forms a Queue of Unpaid Balances (QUB). Initial QUB has the set of unpaid claims that is equal to the sum of the individual capacities of the team members (710). The system then checks to see if the QUB is empty and if yes, nothing is done (730). Otherwise, the first claim is removed from the QUB. A new Claim-Action Pair (CAP) is formed by adding possible actions according to the Set Of Rules (SOR): Correct data in claim; Call adjuster; Return to provider for more info; Write appeal. Afterwards the system computes potential float reduction (PFR) for each claim-action pair. The system then adds new claim-action pairs to the Queue of Unpaid Balances With Actions (QUBWA). The system then reiterates this process until a ranked list of items is created (750).

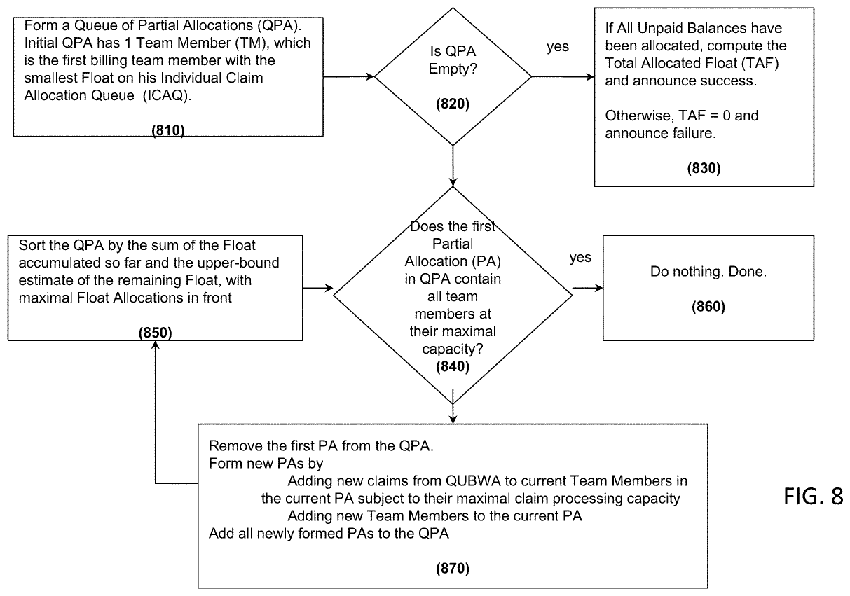

FIG. 8 shows a float chart of another method for a Computer-Assisted Health Insurance Billing To Minimize Payer's Float. In FIG. 8, the processor forms a Queue of Partial Allocations (QPA). The initial QPA has 1 Team Member (TM), which is the first billing team member with the smallest Float on his Individual Claim Allocation Queue (ICAQ) (810).

The system then checks to see if the QPA is empty (820). If yes, then the system checks to see if All Unpaid Balances have been allocated, compute the Total Allocated Float (TAF) and announce success. Otherwise, TAF=0 and announce failure (830).

If the QPA is not empty, the system checks to see if the first Partial Allocation (PA) in QPA contain all team members at their maximal capacity? If yes, then the system does nothing (860). If no, then the system removes the first PA from the QPA and forms new PAs by: adding new claims from QUBWA to current Team Members in the current PA subject to their maximal claim processing capacity; adding new Team Members to the current PA; and adding all newly formed PAs to the QPA (870). The system then sorts the QPA by the sum of the Float accumulated so far and the upper-bound estimate of the remaining Float, with maximal Float Allocations in front (850). The process is reiterated until the Partial Allocation (PA) in QPA contains all team members at their maximal capacity.

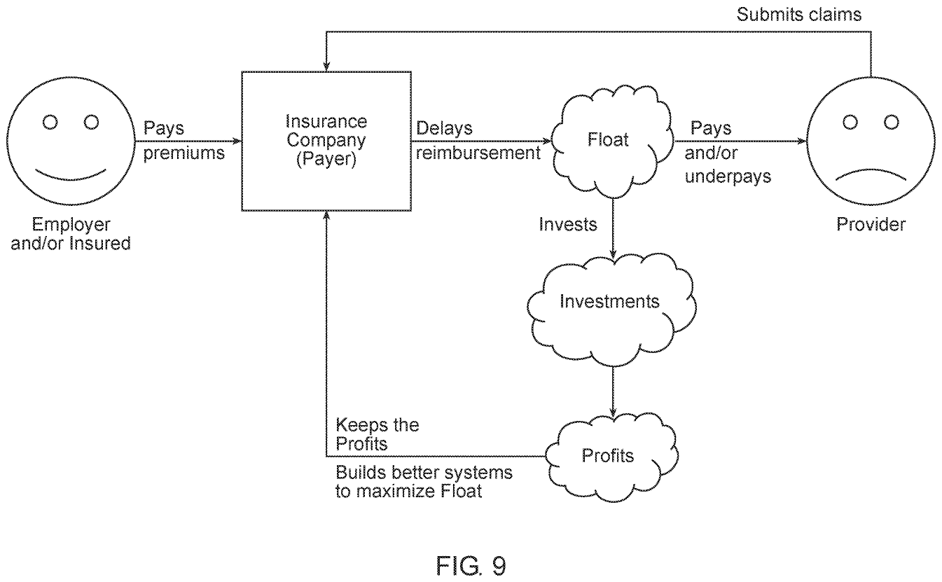

FIG. 9 is a flowchart showing underpayment of an insurance provider.

### Additional Embodiments

Other objects of the invention are achieved by providing computerized methods and system for reducing task complexity of patient practice management.

Other objects of the present invention are achieved by providing a computerized management method for reducing task complexity of patient practice management, the method comprising: software executing on a processor configured to:
communicate with at least one server,
access at least one database to compile a case status list,
generate an action list of tasks that may be performed on said cases, expand said action list to further include all candidates capable of performing said tasks,
prepare said expanded action list for evaluation, wherein unlikely candidates are removed while candidates with a substantial probability of having high potential value are retained,
calculate potential value of said tasks and candidates,
select the highest potential value task and candidate pair, and delegate said selected task to said selected candidate.

In certain embodiments, the potential value is calculated based on minimizing payor float.

In certain embodiments, the delegating said selected task comprises placing said selected task on said selected candidate's workbench.

In certain embodiments, the method includes transmitting said task to the workbench of the selected candidate that is responsible for that function.

In certain embodiments, the delegating said task further comprises providing all relevant information.

In certain embodiments, the delegating said task further comprises providing only relevant information.

In certain embodiments, the method includes comprising reducing errors and delays.

In certain embodiments, the said at least one database is selected from a group consisting of patients, providers, practice management staff, claims, claim validation rules, tasks, process description, or a combination thereof.

Other objects of the invention are achieved by providing a computerized management system useful in delegating tasks, the system comprising:
a processor for accessing at least one database on a server to compile an action list and delegate tasks;
a custom software/algorithm based on game theory used to evaluate potential value of each business action; and
a workbench for receiving delegated tasks based on high potential value.

In certain embodiments, the processor is configured to:
communicate with at least one server,
access at least one database to compile a case status list,
generate an action list of tasks that may be performed on said cases,
expand said action list to further include all candidates capable of performing said tasks,
prepare said expanded action list for evaluation, wherein unlikely candidates are removed while candidates with a substantial probability of having high potential value are retained,
calculate potential value of said tasks and candidates,
select the highest potential value task and candidate pair, and
delegate said selected task to said selected candidate.

In certain embodiments, the system continuously iterates through available databases and selects the best action for a workbench.

It In certain embodiments, the system is in communication with the payor in order to send claims, appeals, notifications and receive notifications and payments.

In certain embodiments, potential value is calculated based on minimizing payor float.

In certain embodiments, potential value is calculated based on minimizing payment delay.

It In certain embodiments, the action list is assembled by acquiring a pending case list, generating tasks for each case, and cross referencing said tasks with capable candidates.

It In certain embodiments, the action list is pruned to remove unlikely candidates identified and selected based on low potential value.

In certain embodiments, the processor compiles a list of all possible actions that may be executed per case.

In certain embodiments, the processor expands the list of possible actions to include alternative candidates for executing said action.

### Embodiments Non-Limiting

What is contemplated by the instant invention is the novel and useful transforming of practice management and payor interaction into a theoretical-game framework; and applying those concepts in the practice management context.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

The disclosed embodiments provide a source array, computer software, and a method for reducing payor float. It should be understood that this description is not intended to limit the embodiments. On the contrary, the embodiments are intended to cover alternatives, modifications, and equivalents, which are included in the spirit and scope of the embodiments as defined by the appended claims. Further, in the detailed description of the embodiments, numerous specific details are set forth to provide a comprehensive understanding of the claimed embodiments. However, one skilled in the art would understand that various embodiments can be practiced without such specific details.

Although the features and elements of aspects of the embodiments are described being in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and can include other examples that occur to those skilled in the art. Such other examples are intended to be within the scope of the claims.

The above-described embodiments are intended to be illustrative in all respects, rather than restrictive, of the embodiments. Thus the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

1. A computer-implemented health insurance billing system for reducing insurance float, the system comprising:
a memory comprising computer executable instructions; and
a processor coupled to the memory and configured by the computer executable instructions, the processor configured to:
(1) access at least one database having patient claim data including a plurality of patient claim data items;
(2) apply a float factor to each of the patient claim data items, the float factor determined by a weighting system adjusted based upon payor data including payor difficulty in making payments, payment delay, and heuristic learning, wherein the weighting system is continuously adjusted based on the payor data;
(3) generate a list of action items based upon the patient claim data items after each of the patient data items has been normalized by the float factor;
(4) sort the list of action items from highest to lowest payor float of each of the patient claim data items, such that the patient claim data items with the highest payor float are given the highest priority for processing;
wherein steps are completed on the processor, and
wherein the computer-implemented health insurance billing system minimizes overall payor float in the system by providing the sorted list of action items to one or more users of the computer-implemented health insurance billing system, such that the one or more users of the computer-implemented health insurance billing system will process the list of action items to process claims and reduce overall insurance float in the system.

2. (canceled)

3. (canceled)

4. (canceled)

5. (canceled)

6. (canceled)

7. (canceled)

8. (canceled)

9. The system of claim 1, wherein the float factor is adjusted based upon batch processing such that patient claim data items are sorted into groups and combinations of groups contribute to payor float.

10. The system of claim 1, wherein the float factor is configured based upon specific rules, the rules being followed to create the ranked list of patient claim data.

11. The system of claim 1, wherein the float factor is adjusted based upon CPT codes and in-network reimbursement fees.

12. The system of claim 1, wherein the computer-implemented health insurance billing system provides a workflow of action items for each day.

13. The system of claim 12, wherein the workflow of action items for each day minimizes overall payor float.

14. The system of claim 1, wherein the system automatically provides a list of action items to work on for each day and delegates them to individual staff at a healthcare institution.

15. The system of claim 1, wherein the system automatically generates a task list on a work bench for multiple users.

16. The system of claim 1, wherein said at least one database is selected from a group consisting of patients, providers, practice management staff, claims, claim validation rules, tasks, process description, or a combination thereof.

17. The system of claim 1, wherein the system is in communication with the payor in order to send claims, appeals, notifications and receive notifications and payments.

18. A computer-implemented method for minimizing overall payor float in a health insurance billing system, the method comprising the steps of:
(1) accessing at least one database having patient claim data including a plurality of patient claim data items;
(2) applying a float factor to each of the patient claim data items, the float factor determined by a weighting system adjusted based upon payor data including payor difficulty in making payments, payment delay, and heuristic learning, wherein the weighting system is continuously adjusted based on the payor; and
(3) generating a list of action items based upon the list of patient claim data items after each of the patient data items has been normalized by the float factor;
(4) sort the list of action items from highest to lowest according to payor float of each of the patient claim data items, such that the patient claim data items with the highest payor float are given the highest priority for processing,
wherein steps (1) and (4) are completed on a processor, and
wherein the computer-implemented health insurance billing system minimizes overall payor float in the system by providing the sorted list of action items to one or more users of the computer-implemented health insurance billing system, such that the one or more users of the computer-implemented health insurance billing system will process the list of action items to process claims and reduce insurance float in the system.

19. (canceled)

20. A non-transitory computer readable storage medium storing a computer program product for minimizing payor float in a healthcare insurance billing system, the non-transitory computer readable storage medium comprising:
computer executable instructions and data, the computer executable instructions able to execute a computer program able to:
(1) access at least one database having patient claim data including a plurality of patient claim data items;
(2) apply a float factor to each of the patient claim data items, the float factor determined by a weighting system adjusted based upon payor data including payor difficulty in making payments, payment delay, and heuristic learning, wherein the weighting system is continuously adjusted based on the payor data;
(3) generate a list of action items based upon the list of patient claim data items after each of the patient data items has been normalized by the float factor;
(4) sort the list of action items from highest to lowest according to payor float of each of the patient claim data items, such that the patient claim data items with the highest payor float are given the highest priority for processing,
wherein steps (1)-(4) are performed on a processor, and
wherein the computer-implemented health insurance billing system minimizes overall payor float in the system by providing the sorted list of action items to one or more users of the computer-implemented health insurance billing system, such that the one or more users of the computer-implemented health insurance billing system will process the list of action items to process claims and reduce insurance float in the system.
